(19) United States
(12) Patent Application Publication
(10) Pub. No.: US 2022/0108785 A1
(43) Pub. Date: Apr. 7, 2022

(54) MEDICAL IMAGE PROCESSING SYSTEM AND METHOD THEREOF

(71) Applicant: MEDICALTEK CO., LTD., Taichung City (TW)

(72) Inventors: Yen-Yu WANG, TAICHUNG CITY (TW); Chieh-Chu CHEN, TAICHUNG CITY (TW); Jing-Ying HUANG, TAICHUNG CITY (TW)

(21) Appl. No.: 17/374,110

(22) Filed: Jul. 13, 2021

(30) Foreign Application Priority Data
Oct. 7, 2020 (TW) 109134740

(51) Int. Cl. G16H 30/20; G16H 10/60

(52) U.S. Cl. CPC G16H 30/20; G16H 10/60

(57) ABSTRACT

The present invention provides a medical image processing method, which comprises following steps of: receiving a three-dimensional model image file and at least one medical record data by a medical image processing device; creating a three-dimensional medical file based on the three-dimensional model image file and the at least one medical record data, wherein the three-dimensional medical file conforms to a digital imaging and communications in medicine standard; transmitting the three-dimensional medical file to a medical picture archiving and communication system; retrieving the three-dimensional medical file from the medical picture archiving and communication system by a three-dimensional medical image browsing device which is a non-workstation computer device loaded with a viewer software; and displaying the three-dimensional medical file.

### FIELD OF THE INVENTION

The invention relates to an image processing system and a method thereof, and more particularly to a medical image processing system and a method thereof

### BACKGROUND OF THE INVENTION

Medical imaging and the processing technology thereof are widely used in medical procedures or medical education. Doctors can better understand a patient's condition and improve the efficiency and accuracy of diagnosis and treatment through viewing and manipulating the medical images.

The establishment of a medical image database through a standardized system is a major issue in medical imaging applications. A good medical image database system can help doctors efficiently obtain the required medical images. However, the functions of the existing medical image data system are imperfect. The existing medical image data system can only displays simple, low-precision and incomplete images, and have limited operation functions for medical images. Therefore, researchers in the art are focus on how to improve the efficiency and accuracy of the medical image data system.

### SUMMARY OF THE INVENTION

The invention provides a medical image processing method and a medical image processing system capable of greatly improving the efficiency and accuracy of medical treatment.

The medical image processing method of the invention comprises following steps of:

building a three-dimensional medical file according to the three-dimensional model image file and the at least one medical record data by the medical image processing device, wherein the three-dimensional medical file conforms to a digital imaging and communications in medicine (DICOM) standard;

transmitting the three-dimensional medical file from the medical image processing device to a medical picture archiving and communication system;

retrieving the three-dimensional medical file from the medical picture archiving and communication system by a three-dimensional medical image browsing device, wherein the three-dimensional medical image browsing device is a non-workstation computer device loaded with a viewer software; and

displaying the three-dimensional medical file by the three-dimensional medical image browsing device.

In one embodiment of the invention, after retrieving the three-dimensional medical file, the medical image processing method further comprises steps of converting the three-dimensional medical file into a temporary file which holds the three-dimensional model image file and the at least one medical record data for the three-dimensional medical image browsing device to display the three-dimensional model image file and the at least one medical record data, and deleting the temporary file after the three-dimensional medical image browsing device is shut down.

In one embodiment of the invention, the three-dimensional model image file comprises a clinical medical image data, and the clinical medical image data includes a plurality of two-dimensional medical images generated by a computed tomography device and/or a nuclear magnetic resonance imaging (NMRI) device.

In one embodiment of the invention, the medical image processing device comprises a spatial information processor and a color visual pattern processor, the spatial information processor retrieves spatial information of the three-dimensional model image file to build a spatial information file, the color visual pattern processor retrieves color visual patterns of the three-dimensional model image file to build a color visual pattern information file, and the medical image processing device builds the three-dimensional medical file based on the at least one medical record data and, the spatial information file and the color visual pattern information file of the three-dimensional model image file.

In one embodiment of the invention, the color visual pattern information file is represented by a matrix showing color data of spatial positions of the three-dimensional model image file.

In one embodiment of the invention, the three-dimensional medical file comprises a core file and a header file, and the medical image processing device comprises a core file generator and a header file generator, the core file generator creates the core file based on the spatial information file and the color visual pattern information file of the three-dimensional model image file, and the header file generator creates the header file based on the at least one medical record data.

In one embodiment of the invention, the at least one medical record data comprises a patient's name, gender, date of birth, medical record number, date of health examination, time of medical treatment, and imaging system information.

In one embodiment of the invention, the three-dimensional medical image browsing device comprises a connector, a three-dimensional medical image decoder, a three-dimensional medical image renderer, a three-dimensional medical image controller and a medical information processor, the connector is connected with the medical picture archiving and communication system to transmit data, the three-dimensional medical image decoder decodes the three-dimensional medical file to generate a spatial information file and a color visual pattern information file of the three-dimensional model image file and the at least one medical record data, the three-dimensional medical image renderer performs image rendering on the spatial information file and the color visual pattern information file of the three-dimensional model image file, the three-dimensional medical image browsing device displays the three-dimensional model image file after image rendering, the three-dimensional medical image controller is provided for moving, zooming, rotating, selecting, and/or measuring angle and distance on the three-dimensional model image file, and the medical information processor is provided for searching, displaying, storing or deleting the three-dimensional medical file.

In one embodiment of the invention, after the three-dimensional medical image browsing device shut down, the three-dimensional medical image browsing device deletes the three-dimensional model image file and the at least one medical record data.

The medical image processing system of the invention comprises a medical image processing device, a medical picture archiving and communication system, and a three-dimensional medical image browsing device. The medical image processing device receives a three-dimensional model image file and at least one medical record data, the medical image processing device building a three-dimensional medical file based on the three-dimensional model image file and the at least one medical record data, wherein the three-dimensional medical file conforms to a digital imaging and communications in medicine (DICOM) standard. The medical image processing device transmits the three-dimensional medical file to the medical picture archiving and communication system. The medical picture archiving and communication system receives the three-dimensional medical file transmitted from the medical image processing device. The three-dimensional medical image browsing device retrieves the three-dimensional medical file from the medical picture archiving and communication system, and displaying the three-dimensional medical file.

Accordingly, the medical image processing method of the invention is to convert the three-dimensional model image file into a file that conforms to the DICOM standard, and temporarily store the file in the browsing device for browsing, wherein the browsing device is a non-workstation computer device loaded with a viewer software. Therefore, the medical image processing method facilitates exchange and browsing of three-dimensional clinical medical images, and allows doctors to view three-dimensional clinical medical images of patients through personal computers wherever he is, thereby improving the efficiency and accuracy of medical treatment.

### BRIEF DESCRIPTION OF THE DRAWINGS

FIG. 1 is a schematic diagram of a medical image processing system according to one embodiment of the invention;

FIG. 2 is a flowchart of a medical image processing method according to one embodiment of the invention; and

FIG. 3 is a schematic diagram of a medical image processing system according to one embodiment of the invention.

### DETAILED DESCRIPTION OF THE PREFERRED EMBODIMENTS

The detailed description, technical contents, features and efficacies of the present invention will be presented in the following detailed description of a preferred embodiment with reference to the drawings. The directional terms mentioned in the following embodiment, for example: above, below, left, right, front or back, etc., are only directions for referring to the attached drawings. Therefore, the directional terms are merely used to illustrate but not to limit the invention.

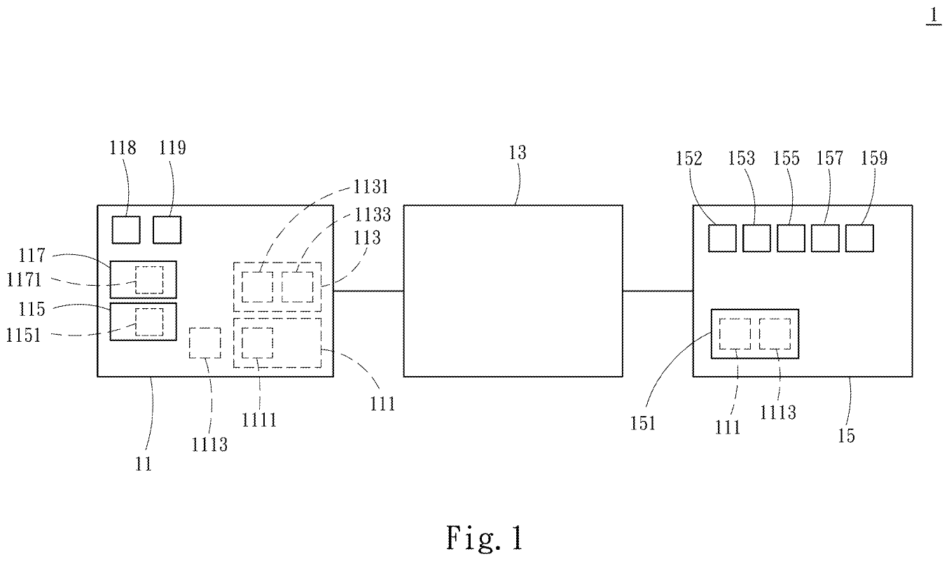

Please refer to FIG. 1 for a schematic diagram of a medical image processing system 1 according to one embodiment of the invention. The medical image processing system 1 comprises a medical image processing device 11, a medical picture archiving and communication system (PACS) 13 and a three-dimensional medical image browsing device 15. The three-dimensional medical image browsing device 15 provides a user, such as a doctor, with three-dimensional medical images and data of patients for browsing and manipulating. The detail operations are described below.

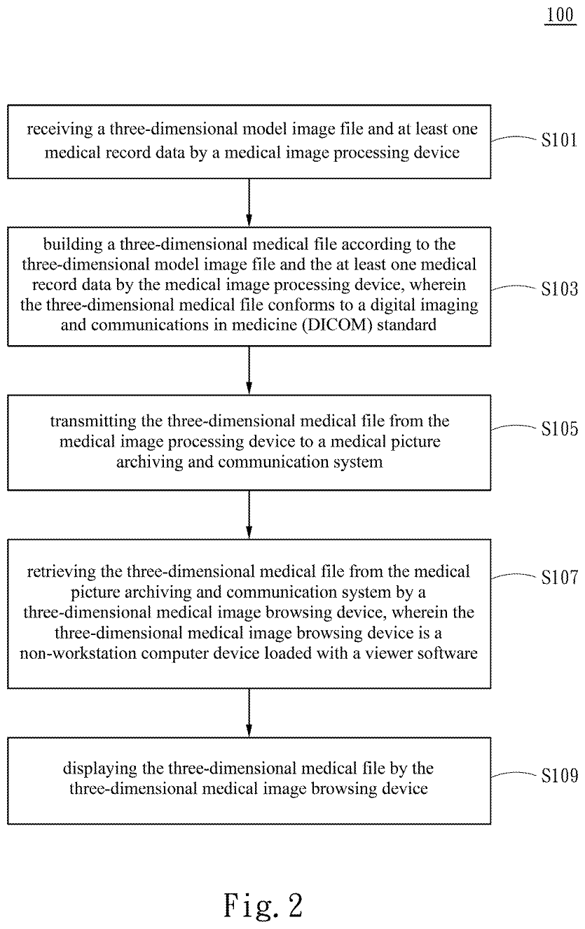

Please refer to FIG. 2 for a flowchart of a medical image processing method 100 according to one embodiment of the invention, wherein the medical image processing method 100 is applied to the medical image processing system 1 as shown in FIG. 1. In step S101, the medical image processing device 11 receives a three-dimensional model image file 111. The three-dimensional model image file 111 comprises a clinical medical image data 1111.

In step S103, the medical image processing device 11 builds a three-dimensional medical file 113 according to the three-dimensional model image file 111 and at least one medical record data 1113. The three-dimensional medical file 113 conforms to a digital imaging and communications in medicine (DICOM) standard.

In step S105, the medical image processing device 11 transmits the three-dimensional medical file 113 to the medical picture archiving and communication system 13. Since the three-dimensional medical file 113 conforms to the DICOM standard, the three-dimensional medical file 113 is stored in the medical picture archiving and communication system 13. In step S107, the three-dimensional medical image browsing device 15 retrieves the three-dimensional medical file 113 from the medical picture archiving and communication system 13. In step S109, the three-dimensional medical image browsing device 15 displays the three-dimensional medical file 113.

Further, after retrieving the three-dimensional medical file 113, the medical image processing method 100 further comprises a step of converting the three-dimensional medical file 113 into a temporary file 151 in the three-dimensional medical image browsing device 15 to protect privacy of patients. The temporary file 151 holds the three-dimensional model image file 111 and the at least one medical record data 1113 for three-dimensional medical image browsing device 15 to display the three-dimensional model image file 111 and the at least one medical record data 1113. Then, the temporary file 151 is deleted after browsing is finished. For example, while the three-dimensional medical image browsing device 15 is closed with the temporary file 151, the temporary file 151 is deleted. Therefore, no data will be left in the three-dimensional medical image browsing device 15, and the privacy of patients can be protected. In one embodiment, the three-dimensional medical image browsing device 15 is a non-workstation computer device, such as a personal computer, a tablet computer and a smart phone, with a viewer software. Signal transmission among the medical image processing device 11, the medical picture archiving and communication system 13, and the three-dimensional medical image browsing device 15 can be wired or wireless transmission, and the invention is not limited thereto.

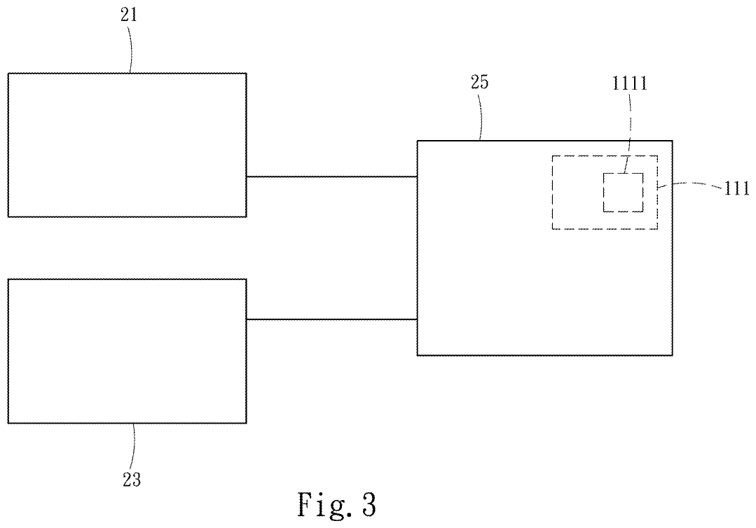

Please refer to FIG. 3 for a schematic diagram of a medical image processing system 1 according to one embodiment, particularly for illustrating the construction of the three-dimensional model image file 111 with the clinical medical image data 1111. In one embodiment, the clinical medical image data 1111 includes a plurality of two-dimensional medical images generated by a computed tomography device 21 and/or a nuclear magnetic resonance imaging (NMRI) device 23 during clinical treatment on a patient.

As shown in FIG. 3, an image processing device 25 performs image processing on the clinical medical image data 1111 to construct the three-dimensional model image file 111. In one embodiment, the image processing device 25 performs volume rendering on the clinical medical image data 1111 to construct the three-dimensional model image file 111. The image processing device 25 is implemented by a computer device or a workstation. In one embodiment, the medical image processing device 11 comprises the image processing device 25; that is, the image processing device 25 is built in the medical image processing device 11.

Please refer to FIG. 1. The medical image processing device 11 further comprises a spatial information processor 115 and a color visual pattern processor 117. The spatial information processor 115 retrieves spatial information of the three-dimensional model image file 111 to build a spatial information file 1151. The color visual pattern processor 117 retrieves color visual patterns of the three-dimensional model image file 111 to build a color visual pattern information file 1171. The medical image processing device 11 builds the three-dimensional medical file 113 conforming to the DICOM standard according to the spatial information file 1151 and the color visual pattern information file 1171 of the three-dimensional model image file 111 and the at least one medical record data 1113. Further, the color visual pattern information file 1171 is represented by a matrix (not shown in the figures) showing color data of spatial positions of the three-dimensional model image file 111. The medical image processing device 11 builds the three-dimensional medical file 113 based on the spatial information file 1151 and the color visual pattern information file 1171 represented by the matrix.

In detail, the three-dimensional medical file 113 comprises a core file 1131 and a header file 1133. The core file 1131 comprises spatial information and color information of the three-dimensional model image file 111, and the header file 1133 comprises medical record. The medical image processing device 11 comprises a core file generator 118 and a header file generator 119. The core file generator 118 creates the core file 1131 according to the spatial information file 1151 and the color visual pattern information file 1171 of the three-dimensional model image file 111, and the header file generator 119 creates the header file 1133 according to the at least one medical record data 1113. Thereby, the three-dimensional medical file 113 created by the medical image processing device 11 conforms to the DICOM standard.

In one embodiment, the at least one medical record data 1113 includes a patient's name, gender, date of birth, medical record number, date of health examination, time of medical treatment, and imaging system information. The imaging system information comprises information of the clinical medical image data 1111 created by equipments such as computed tomography device and nuclear magnetic resonance imaging device.

The three-dimensional medical image browsing device 15 comprises a connector 152, a three-dimensional medical image decoder 153, a three-dimensional medical image renderer 155, a three-dimensional medical image controller 157, and a medical information processor 159. The connector 152 is connected with the medical picture archiving and communication system 13 to transmit data. The three-dimensional medical image decoder 153 decodes the three-dimensional medical file 113 to generate the spatial information file 1151 and the color visual pattern information file 1171 of the three-dimensional model image file 111, and the at least one medical record data 1113. The three-dimensional medical image renderer 155 performs image rendering on the spatial information file 1151 and the color visual pattern information file 1171 of the three-dimensional model image file 111. In one embodiment, the three-dimensional medical image browsing device 15 displays the three-dimensional model image file 111 after image rendering. The three-dimensional medical image controller 157 is provided for moving, zooming, rotating, selecting, and/or measuring angle and distance on the three-dimensional model image file 111.Therefore, the three-dimensional medical image controller 157 is provided to a user, such as a doctor, for manipulating the three-dimensional model image file 111. The medical information processor 159 is provided for searching, displaying, storing or deleting the three-dimensional medical file 113.

Moreover, the temporary file 151 converted from the three-dimensional medical image browsing device 15 holds information of the three-dimensional model image file 111 and the at least one medical record data 1113 that are decoded and generated by the three-dimensional medical image decoder 153.

In one embodiment, after the three-dimensional medical image browsing device 15 is shut down, the three-dimensional medical image browsing device 15 deletes the three-dimensional model image file 111 and the at least one medical record data 1113.

In summary, the method of the invention is to construct the three-dimensional model image file that conforms to the DICOM standard, and temporarily store the file in the browsing device for browsing, wherein the browsing device is a non-workstation computer device loaded with a viewer software. Thus, the medical image processing method facilitates exchange and browsing of three-dimensional clinical medical images, and allows doctors to view three-dimensional clinical medical images of patients through personal computers wherever he is, thereby improving the efficiency and accuracy of medical treatment.

What is claimed is:

1. A medical image processing method, comprising following steps of:
receiving a three-dimensional model image file and at least one medical record data by a medical image processing device;
building a three-dimensional medical file according to the three-dimensional model image file and the at least one medical record data by the medical image processing device, wherein the three-dimensional medical file conforms to a digital imaging and communications in medicine (DICOM) standard;
transmitting the three-dimensional medical file from the medical image processing device to a medical picture archiving and communication system;
retrieving the three-dimensional medical file from the medical picture archiving and communication system by a three-dimensional medical image browsing device, wherein the three-dimensional medical image browsing device is a non-workstation computer device loaded with a viewer software; and
displaying the three-dimensional medical file by the three-dimensional medical image browsing device.

2. The medical image processing method as claimed in claim 1, wherein after retrieving the three-dimensional medical file, the medical image processing method further comprises steps of converting the three-dimensional medical file into a temporary file which holds the three-dimensional model image file and the at least one medical record data for the three-dimensional medical image browsing device to display the three-dimensional model image file and the at least one medical record data, and deleting the temporary file after the three-dimensional medical image browsing device is shut down.

3. The medical image processing method as claimed in claim 1, wherein the three-dimensional model image file comprises a clinical medical image data, and the clinical medical image data includes a plurality of two-dimensional medical images generated by a computed tomography device and/or a nuclear magnetic resonance imaging (NMRI) device.

4. The medical image processing method as claimed in claim 1, wherein the medical image processing device comprises a spatial information processor and a color visual pattern processor, the spatial information processor retrieves spatial information of the three-dimensional model image file to build a spatial information file, the color visual pattern processor retrieves color visual patterns of the three-dimensional model image file to build a color visual pattern information file, and the medical image processing device builds the three-dimensional medical file based on the at least one medical record data and, the spatial information file and the color visual pattern information file of the three-dimensional model image file.

5. The medical image processing method as claimed in claim 4, wherein the color visual pattern information file is represented by a matrix showing color data of spatial positions of the three-dimensional model image file.

6. The medical image processing method as claimed in claim 4, wherein the three-dimensional medical file comprises a core file and a header file, and the medical image processing device comprises a core file generator and a header file generator, the core file generator creates the core file based on the spatial information file and the color visual pattern information file of the three-dimensional model image file, and the header file generator creates the header file based on the at least one medical record data.

7. The medical image processing method as claimed in claim 1, wherein the at least one medical record data comprises a patient's name, gender, date of birth, medical record number, date of health examination, time of medical treatment, and imaging system information.

8. The medical image processing method as claimed in claim 1, wherein the three-dimensional medical image browsing device comprises a connector, a three-dimensional medical image decoder, a three-dimensional medical image renderer, a three-dimensional medical image controller and a medical information processor, the connector is connected with the medical picture archiving and communication system to transmit data, the three-dimensional medical image decoder decodes the three-dimensional medical file to generate a spatial information file and a color visual pattern information file of the three-dimensional model image file and the at least one medical record data, the three-dimensional medical image renderer performs image rendering on the spatial information file and the color visual pattern information file of the three-dimensional model image file, the three-dimensional medical image browsing device displays the three-dimensional model image file after image rendering, the three-dimensional medical image controller is provided for moving, zooming, rotating, selecting, and/or measuring angle and distance on the three-dimensional model image file, and the medical information processor is provided for searching, displaying, storing or deleting the three-dimensional medical file.

9. The medical image processing method as claimed in claim 8, wherein after the three-dimensional medical image browsing device shut down, the three-dimensional medical image browsing device deletes the three-dimensional model image file and the at least one medical record data.

10. A medical image processing system, comprising:
a medical image processing device, receiving a three-dimensional model image file and at least one medical record data, the medical image processing device building a three-dimensional medical file based on the three-dimensional model image file and the at least one medical record data, wherein the three-dimensional medical file conforms to a digital imaging and communications in medicine (DICOM) standard;
a medical picture archiving and communication system, receiving the three-dimensional medical file transmitted from the medical image processing device; and
a three-dimensional medical image browsing device, retrieving the three-dimensional medical file from the medical picture archiving and communication system, and displaying the three-dimensional medical file.
